(19) United States
(12) Patent Application Publication
(10) Pub. No.: US 2023/0210772 A1
(43) Pub. Date: Jul. 6, 2023

(54) METHODS OF DECREASING MUSCLE ATROPHY AND/OR PROMOTING MUSCLE REGENERATION

(71) Applicant: ABBOTT LABORATORIES, Abbott Park, IL (US)

(72) Inventors: José María LÓPEZ PEDROSA, Cenes de la Vega (ES); Jorge GARCÍA MARTÍNEZ, Armilla (ES)

(21) Appl. No.: 17/996,084

(22) PCT Filed: Apr. 19, 2021
(86) PCT No.: PCT/US2021/027915

§ 371 Date: Oct. 13, 2022

(30) Foreign Application Priority Data
Apr. 20, 2020 (EP) 20382316.6

(51) Int. Cl. A61K 9/127; A61K 35/20; A61P 21/06; A23L 33/00; A23L 33/19; A23L 33/185; A23L 33/115; A23L 33/125

(52) U.S. Cl. CPC A61K 9/1276; A61K 35/20; A61P 21/06; A23L 33/40; A23L 33/19; A23L 33/185; A23L 33/115; A23L 33/125

(57) ABSTRACT

Methods of decreasing muscle atrophy and/or promoting muscle regeneration in a subject at risk of muscle atrophy comprise orally administering a nutritional composition comprising at least one of protein, fat and carbohydrate, and bovine milk-isolated exosomes comprising intact exosomes. In specific embodiments, the subject suffers from malnutrition, acquired immune deficiency syndrome (AIDS), cancer, diabetes, chronic obstructive pulmonary disease (COPD), amyotrophic lateral sclerosis (ALS), non-alcoholic fatty liver disease (NAFLD), or a burn injury, or has undergone clinical corticosteroid treatment.

### FIELD

The present invention is directed to methods of decreasing muscle atrophy and/or promoting muscle regeneration by orally administering a nutritional composition comprising bovine milk-isolated exosomes comprising intact exosomes.

### BACKGROUND

Skeletal muscle is the most abundant tissue in the body. The mass and functionality of skeletal muscle are key determinants of strength, endurance and physical performance throughout a lifespan. Skeletal muscle is a plastic tissue that shows variations in muscle mass and muscle fiber size according to physiological and pathological conditions.

Skeletal muscle mass is maintained by a delicate balance between protein synthesis and protein degradation. The muscle is a highly adaptive tissue that responds rapidly to anabolic stimuli, such as physical activity or food intake. Conversely, prolonged fasting or immobilization is known to cause rapid muscle loss. Muscle atrophy occurs when protein degradation rates exceed protein synthesis. This phenomenon happens in a wide variety of conditions. For instance, muscle wasting is a common trait which has been associated with poor prognosis and negative outcomes in diseases such as acquired immune deficiency syndrome (AIDS), cancer, diabetes, chronic obstructive pulmonary disease (COPD), amyotrophic lateral sclerosis (ALS), non-alcoholic fatty liver disease (NAFLD), and burn injuries. A catabolic condition, in which muscle loss is favored over muscle growth, also occurs during the human lifespan. From the age of 35-40 years in adult humans, muscle mass typically starts to decline progressively by 0.4-1.0% per year. The age-related loss of muscle mass and strength in otherwise healthy, aging individuals is referred to as sarcopenia. The resulting decline in muscle mass and strength may increase the risk of developing metabolic diseases and/or physical disabilities and can result in the inability to maintain daily functioning. There is a general consensus among the scientific community that muscle atrophy is associated with a variety of undesirable outcomes, including delayed recovery from illness, poorer quality of life, physical disability, reduced resting metabolic rate, decreased insulin sensitivity, slowed wound healing, and higher health care costs.

Skeletal muscle tissue responds to anabolic stimuli, i.e., dietary protein intake and physical activity, for protein synthesis. Nevertheless, for subjects encountering injury, illness, and/or aging, implementation of sufficient physical activity for protein synthesis to maintain or increase muscle mass is not always possible. Therefore, it would be desirable to develop nutritional intervention strategies to resist or reduce the loss of muscle mass and strength and/or to promote muscle regeneration.

### SUMMARY

The present invention is directed to methods of decreasing muscle atrophy and/or promoting muscle regeneration in a subject at risk of muscle atrophy. It is an object of the invention to provide such methods suitable for, among others, subjects for whom implementation of physical activity intervention sufficient for protein synthesis to maintain or increase muscle mass is not convenient and/or possible.

In one embodiment, the invention is directed to methods of decreasing muscle atrophy and/or promoting muscle regeneration in a subject at risk of muscle atrophy. The methods comprise orally administering a nutritional composition comprising at least one of protein, fat and carbohydrate, and bovine milk-isolated exosomes comprising intact exosomes.

The methods of the invention are advantageous in providing a convenient manner to reduce muscle atrophy and/or promote muscle regeneration in a subject at risk of muscle atrophy. The methods may be conducted on a continual basis over a period of time as needed depending on the risk of the subject to muscle atrophy. These and additional advantages of the inventive methods will be more fully apparent in view of the detailed description.

### BRIEF DESCRIPTION OF THE DRAWINGS

Certain aspects of the invention are illustrated in the drawings, in which:

FIG. 1 shows that protein degradation by dexamethasone (C) and in the presence of intact bovine milk-isolated exosomes (Ex) as described in Example 2;

FIG. 2 shows Akt phosphorylation of myotubes incubated alone (C) and with bovine milk-isolated exosomes as described in Example 2;

FIG. 3 shows the effects of various components on the transcriptional activity of the ubiquitin promoter, as described in Example 2;

FIG. 4 shows the effects of various components on the atrogin-1 protein level, as described in Example 2;

FIG. 5 shows the effects of various components on FoxO transcriptional activity, as described in Example 2; and

FIG. 6 shows the effects of intact bovine milk-isolated exosomes and sonicated exosomes, respectively, on Mef2 in myoblasts, as described in Example 2.

### DETAILED DESCRIPTION

While the general inventive concepts are susceptible of embodiment in many different forms, described herein in detail are specific embodiments of the invention, with the understanding that the present disclosure is to be considered as an exemplification of the principles of the general inventive concepts. Accordingly, the general inventive concepts are not intended to be limited to the specific embodiments illustrated and described herein.

In one embodiment, the invention is directed to methods which involve administering nutritional compositions. The term “nutritional composition” as used herein, unless otherwise specified, encompasses all forms of nutritional compositions, including nutritional liquids, including emulsified liquids, and liquids formed by reconstituting nutritional powders, for example, by addition of water, and nutritional solids, including, but not limited to those in powder form. The nutritional compositions are suitable for oral consumption by a human.

All percentages, parts and ratios as used herein are by weight of the total composition, unless otherwise specified. All such weights as they pertain to listed ingredients are based on the active level and, therefore, do not include solvents or byproducts that may be included in commercially available materials, unless otherwise specified.

The terminology as set forth herein is for description of the embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

Throughout this specification, when a range of values is defined with respect to a particular characteristic of the present invention, the present invention relates to and explicitly incorporates every specific subrange therein. Additionally, throughout this specification, when a group of substances is defined with respect to a particular characteristic of the present invention, the present invention relates to and explicitly incorporates every specific subgroup therein. Any specified range or group is to be understood as a shorthand way of referring to every member of a range or group individually as well as every possible subrange or subgroup encompassed therein.

The various embodiments of the nutritional compositions employed in the methods of the present disclosure may also be substantially free of any optional or selected ingredient or feature described herein, provided that the remaining nutritional composition still contains all of the required ingredients or features as described herein. In this context, and unless otherwise specified, the term “substantially free” means that the selected nutritional product contains less than a functional amount of the optional ingredient, typically less than 1%, including less than 0.5%, including less than 0.1%, and also including zero percent, by weight, of such optional or selected essential ingredient.

The methods and nutritional compositions described herein may comprise, consist of, or consist essentially of the essential steps and elements, respectively, as described herein, as well as any additional or optional steps and elements, respectively, described herein. Any combination of method or process steps as used herein may be performed in any order, unless otherwise specified or clearly implied to the contrary by the context in which the referenced combination is made.

Unless otherwise indicated herein, all exemplary embodiments, sub-embodiments, specific embodiments and optional embodiments are respective exemplary embodiments, sub-embodiments, specific embodiments and optional embodiments to all embodiments described herein.

In one embodiment, the invention is directed to a method of decreasing muscle atrophy and/or promoting muscle regeneration in a subject at risk of muscle atrophy. In a specific embodiment, the subject is a human. In other embodiments, the subject is a non-human animal. For example, the subject may be an aging human adult, for example, over 40 years of age, over 45 years of age, over 50 years of age, over 55 years of age, over 60 years of age, over 65 years of age, over 70 years of age, or older. As discussed previously, aging adults typically exhibit some reduction in muscle mass and may encounter difficulties in preventing such a reduction by dietary protein intake and exercise alone. The age-related loss of muscle mass and strength in otherwise healthy, older individuals is also referred to as sarcopenia.

In additional embodiments, the subject may be experiencing an event that involves, or involves a risk of developing, muscle atrophy. For example, the subject may suffer from acquired immune deficiency syndrome (AIDS), cancer, including cancer cachexia, diabetes, chronic obstructive pulmonary disease (COPD), amyotrophic lateral sclerosis (ALS), non-alcoholic fatty liver disease (NAFLD), or a burn injury, which conditions typically involve muscle wasting. In additional embodiments, the subject may have experienced malnutrition for an extended period of time and/or undergone clinical corticosteroid treatment.

The inventive methods comprise orally administering a nutritional composition comprising at least one of protein, fat and carbohydrate, and bovine milk-isolated exosomes comprising intact exosomes. The inventors have surprisingly discovered that intact bovine milk-isolated exosomes influence certain cellular mechanisms which contribute to reduce muscle atrophy and/or contribute to increase muscle regeneration.

The term “bovine milk-isolated exosomes” as used herein, unless otherwise specified, refers to exosomes that have been substantially separated from other bovine milk components such as lipids, cells, and debris, and are concentrated in an amount higher than that found in bovine milk. Milk exosomes are small solid particles that are “dissolved” in bovine milk and account for a minor percentage of milk's total solids. Isolation of the exosomes as described herein produces a fluid in which the exosomes originally present in milk are concentrated. As will be apparent, the bovine milk-isolated exosomes may also contain other milk solids that share a size with the milk exosomes and are co-isolated with the exosomes (i.e., caseins and other whey proteins). The term “powdered exosomes” as used herein, unless otherwise specified, refers to a dry powder that contains exosomes which have been isolated from bovine milk. The isolated exosomes are dried to form a dry powder. As the isolated fluid containing the exosomes also contains co-isolated milk solids as described above, the powdered exosomes also contain such other milk solids in the resulting powder. In one embodiment, the isolated exosomes contain at least 10 wt % exosomes, at least 15 wt % exosomes, at least 20 wt %, or at least 25 wt % exosomes, and a balance of other bovine milk-isolated components.

Importantly, the powdered exosomes of the invention comprise intact exosomes. An intact exosome is one in which the lipid membrane is undamaged and the contents of the exosome are retained within the exosome. Intact bovine milk exosomes contain various bioactive agents, for example, multiple miRNAs for promoting healthy function of diverse organs, tissues, and systems. However, if the lipid membrane of an exosome is ruptured, factors such as miRNAs tend to degrade quickly and their beneficial functions are lost. Milk exosomes provide a protective environment for miRNAs, but many current techniques for isolating exosomes often lead to damage to the exosome membrane and consequently degradation of the bioactive agents. The present invention employs intact isolated exosomes which are obtained from bovine milk using gentle procedures which do not disrupt the exosome membrane, thereby leaving the exosome intact and the bioactive agents contained within the exosome structure.

Although various methods may be employed to confirm that exosomes are intact, one such method employs uranyl acetate staining, for example, staining with 2% uranyl acetate for 5 minutes. Uranyl acetate “negatively” stains exosomes, i.e., uranyl acetate stains the inner compartment of the exosome only when the exosome membrane is broken or damaged. In addition, exosomes in which the membrane is damaged tend to aggregate and lose their typical spherical shape, for example, when viewed using transmission electron microscopy (TEM) at 10,000×.

In one embodiment, isolated and concentrated exosomes are provided in a liquid suspension. In another embodiment, a suspension of concentrated exosomes is dried to provide powdered exosomes. Various methods may be employed to isolate exosomes with care being exercised to avoid disruption of the lipid membrane. Fresh bovine milk, refrigerated bovine milk, thawed frozen bovine milk, or otherwise preserved bovine milk may be employed as a source of exosomes. In specific embodiments, isolating the exosomes comprises performing the isolation immediately upon obtaining milk from a bovine. In additional embodiments, isolating the exosomes comprises performing the isolation within about 1 day, or about 2 days, or about 3 days, or about 4 days, or about 5 days or about 6 days, or about 7 days from the time of obtaining the milk from a bovine. In specific embodiments, the exosomes are isolated within about 10 days, or within about 14 days from the time of obtaining milk from a bovine. In additional embodiments, the bovine milk may be frozen and then thawed for processing for isolating exosomes, with the bovine milk preferably having been frozen within about 1 day, or about 2 days, or about 3 days, or about 4 days, or about 5 days or about 6 days, or about 7 days from the time of obtaining the milk from a bovine. Thawed milk is preferably processed immediately upon thawing. In a specific embodiment, fresh bovine milk is subjected to the processing within about 5 days of obtaining the milk from a bovine, or thawed bovine milk which is subjected to processing is thawed from bovine milk that was frozen within about 5 days of obtaining the milk from a bovine.

In one embodiment, a whey-containing bovine milk fraction or, specifically, cheese whey, serves as a source of exosomes. In a specific embodiment, the whey-containing bovine milk fraction is provided by lowering the pH of a bovine milk product, for example, to about 3.0 to 4.6, to precipitate milk solids, and removing the milk solids. Such a fraction is often produced as a by-product in cheese-making and is referred to as cheese whey.

In one embodiment, exosomes are isolated from bovine milk or a bovine milk product such as cheese whey by centrifuging the bovine milk or bovine milk product to form a lipid fraction top layer, a whey fraction middle layer, and a first pellet of cells and debris. The whey fraction is separated from the lipid fraction and the first pellet and is subjected to one or more further centrifugations, for example at higher speeds and, optionally, for increased times, to produce a substantially clear whey fraction. Additional fat, casein aggregates, and debris, for example, are removed to produce the substantially clear whey fraction. The substantially clear whey fraction is then microfiltered to remove residual debris. The microfiltered whey fraction is then subjected to additional centrifugation to obtain a second pellet containing exosomes. This second pellet is then carefully suspended in aqueous medium to dissolve the second pellet without disrupting the membrane of exosomes therein and to provide an exosome suspension. It is important to suspend the second pellet in a gentle manner which does not disrupt the exosome membrane. In one specific embodiment, the second pellet is incubated for an extended period of time, for example at least 6 hours, at least 8 hours, at least 10 hours or at least 12 hours, and up to 18, 24, 30 or 36, or more, hours, in an aqueous medium such as sterile phosphate buffered saline (PBS) or water. For example, an orbital shaker at low speed, i.e., not more than 500 rpm, may be employed. Once the pellet is fully suspended, the suspension can be added to a nutritional composition in suspension form or may be dried to obtain powdered exosomes. Again, any drying step must be conducted with care to avoid disruption of the lipid membrane of the exosomes. In one specific embodiment, the drying step comprises freeze drying.

In specific embodiments of the invention, exosome isolation comprises centrifugation at specific speeds, times and/or temperatures. Centrifugation times and speeds as described herein provide for intact exosome isolation, as centrifuging can cause exosome membrane damage if performed too forcefully. In specific embodiments, bovine milk is centrifuged below about 15,000 G, for example, at about 12,000 G, for example, at about 4° C. for about 15 minutes, to obtain a whey layer formed between a top layer of fat (lipid) and the cell debris pellet. In specific embodiments, the whey fraction is centrifuged two more times, again under conditions to maintain the exosomes in intact form, for example, at about 21,000 G and about 4° C. for about 30 minutes, each time to remove additional fat and/or debris. A substantially clear whey fraction is obtained. The substantially clear whey fraction is microfiltered, for example, using a 0.22 μm filter of hydrophilic material such as polyether sulfone having low protein retention, and the microfiltered whey is then centrifuged at 100,000 G at 4° C. for about 60 minutes, to form an exosome-containing pellet. In specific embodiments, the exosome-containing pellet is dissolved in an orbital the shaker by incubation for at least about 12 hours, or from about 12 hours to about 36 hours, or from about 15 hours to about 30 hours, or from about 18 hour to about 24 hours. The dissolution is conducted under conditions which maintain the exosomes in intact form, i.e., the membrane is not damaged and the contents of the exosomes are maintained therein.

The exosomes may be dried if a powder form is desired for storage or handling, or for addition to a composition with other ingredients, for example, a nutritional composition. Any such drying is conducted under conditions which maintain the exosomes in intact form. In specific embodiments of the invention, the isolated exosomes are dried, for example by freeze-drying or spray drying, to form powdered exosomes under conditions which maintain the exosomes in intact form. In specific embodiments of the invention, the step of freeze-drying comprises exposing the exosome suspension to a temperature of −80° C. and a vacuum of less than 0.3 mbar for a sufficient time period. In specific embodiments, depending on the amount of liquid to be freeze-dried and the features of the freeze-drying equipment, the time may vary from about 5 to about 40 hours, more specifically from about 10 to about 30 hours, or from about 15 to about 25 hours. Importantly, the process should fully dry the exosomes. In a specific embodiment as described in the examples, at a temperature of −80° C. and a vacuum of less than 0.3 mbar, the time period for freeze-drying was 24 hours or more.

Additional embodiments for providing intact bovine milk-isolated exosomes for use in the methods of the invention comprise variations of time, temperature, and pressure for freeze-drying. In additional embodiments of the method, the milk exosomes are maintained at a temperature of at least about −50° C., or at least about −60° C., or at least about −70° C., or at least about −80° C., are subjected to a vacuum of less than about 0.3 mbar, or less than about 0.2 mbar, or less than about 0.1 mbar, and maintained under these conditions for at least about 5, 10, 15, 20, 25, 30, 35 or 40 hours.

Both liquid suspensions of exosomes and powdered exosomes produced according to such methods comprise intact exosomes, i.e., exosomes in which the membrane is not ruptured and/or otherwise degraded and the contents of the exosomes are retained therein. In specific embodiments, at least about 50 wt % of the exosomes in an exosome suspension or powdered form are intact. In further embodiments, at least about 55, 60, 65, 70, 75, 80, 85, 90, or 95 wt % of the exosomes in an exosome suspension or powdered form are intact.

In specific embodiments, greater than 90% of the isolated exosomes are from about 10 nanometers to about 250 nanometers in diameter, or from about 20 to 200 nm in diameter, or from about 50 to 150 nm in diameter.

Specific embodiments of nutritional compositions described herein include a protein, a carbohydrate, and/or a fat, and bovine milk-isolated exosomes. The bovine milk-isolated exosomes may be included in the nutritional compositions in any desired amount effective to provide the described therapeutic benefit of reducing muscle atrophy and/or increasing muscle regeneration. In a specific embodiment, the nutritional compositions comprise from about 0.001 to about 10 wt % of the bovine milk-isolated exosomes, in liquid suspension or powdered form, or, more specifically, from about 0.1 to about 5 wt %, of the bovine milk-isolated exosomes, based on the weight of the composition. All references to the amounts of bovine milk-isolated exosomes in a composition refers to the amount of liquid suspension or powder containing the exosomes which is added to the composition.

A wide variety of sources and types of protein, carbohydrate, and/or fat can be used in embodiments of nutritional compositions described herein. In specific embodiments, the nutritional compositions employed in the present methods comprise protein, carbohydrate and fat.

In specific embodiments of the nutritional composition, protein comprises from about 1 wt % to about 30 wt % of the nutritional composition. In more specific embodiments, the protein comprises from about 1 wt % to about 25 wt % of the nutritional composition, including about 1 wt % to about 20 wt %, about 1 wt % to about 15 wt %, about 1 wt % to about 10 wt %, about 5 wt % to about 10 wt %, or about 10 wt % to about 20 wt % of the nutritional composition. In additional specific embodiments, the protein comprises from about 1 wt % to about 5 wt % of the nutritional composition. In additional, specific embodiments, the protein comprises from about 20 wt % to about 30 wt % of the nutritional composition.

One or more proteins may be included in the nutritional composition. A wide variety of sources and types of protein can be used in the nutritional compositions which are employed in the methods of the invention. For example, the source of protein may include, but is not limited to, intact, hydrolyzed, and partially hydrolyzed protein, which may be derived from any suitable source such as milk (e.g., casein, whey), animal (e.g., meat, fish), cereal (e.g., rice, brown rice, corn, barley, etc.), vegetable (e.g., soy, pea, yellow pea, fava bean, chickpea, canola, potato, mung, ancient grains such as quinoa, amaranth, and chia, hamp, flax seed, etc.), and combinations of two or more thereof. The protein may also include one or a mixture of amino acids (often described as free amino acids) known for use in nutritional products, and/or metabolites thereof, or a combination of one or more such amino acids and/or metabolites, with the intact, hydrolyzed, and partially hydrolyzed proteins described herein. The amino acids may be naturally occurring or synthetic amino acids.

More specific examples of proteins which are suitable for use in the exemplary nutritional compositions described herein include, but are not limited to, whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins such as beef protein isolate and/or chicken protein isolate, potato proteins, chickpea proteins, canola proteins, mung proteins, quinoa proteins, amaranth proteins, chia proteins, hamp proteins, flax seed proteins, earthworm proteins, insect proteins, and combinations of two or more thereof. Suitable amino acids may be naturally occurring or synthetic amino acids. In one embodiment, one or more branched chain amino acids (leucine, isoleucine and/or valine) and/or one or more metabolites of branched chain amino acids, for example, leucic acid (also known as α-hydroxyisocaproic acid or HICA), keto isocaproate (KIC), and/or β-hydroxy-β-methylbutyrate (HMB), are included as a protein in the nutritional compositions. The nutritional compositions can include any individual source of protein or combination of any of the various sources of protein listed above.

In specific embodiments, carbohydrate is present in an amount from about 5 wt % to about 75 wt % of the nutritional composition. In more specific embodiments, the carbohydrate is present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, or about 15 wt % to about 25 wt %, of the nutritional composition.

Carbohydrates suitable for use in the nutritional compositions may be simple or complex, or variations, or combinations thereof. Various sources of carbohydrate may be used so long as the source is suitable for use in a nutritional composition and is otherwise compatible with any other selected ingredients or features present in the nutritional composition. Non-limiting examples of carbohydrates suitable for use in the nutritional compositions include maltodextrin, hydrolyzed or modified starch, hydrolyzed or modified cornstarch, glucose polymers such as polydextrose and dextrins, corn syrup, corn syrup solids, rice-derived carbohydrates such as rice maltodextrin, brown rice mild powder and brown rice syrup, sucrose, glucose, fructose, lactose, high fructose corn syrup, honey, sugar alcohols (e.g., maltitol, erythritol, sorbitol), isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, Fibersol™, fruit puree, vegetable puree, isomalto-oligosaccharides, monosaccharides, disaccharides, tapioca-derived carbohydrates, inulin, other digestion-resistant starches, and artificial sweeteners, and combinations of two or more thereof. The nutritional compositions may include any individual source of carbohydrate or combination of any of the various sources of carbohydrate listed above.

The term “fat” as used herein, unless otherwise specified, refers to lipids, fats, oils, and combinations thereof. In specific embodiments, the nutritional composition comprises about 0.5 wt % to 20 wt % of the nutritional composition. In more specific embodiments, the fat comprises about 0.5 wt % to 18 wt % of the nutritional composition, including about 0.5 wt % to 15 wt %, about 0.5 wt % to 10 wt %, about 0.5 wt % to 5 wt %, about 2 wt % to 8 wt %, about 5 wt % to 10 wt %, about 8 wt % to 12 wt %, or about 12 wt % to 18 wt % of the nutritional composition.

Fats suitable for use in the nutritional composition include, but are not limited to, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, and long chain polyunsaturated fatty acids such as docosahexanoic acid (DHA), arachidonic acid (ARA), docosapentaenoic acid (DPA), eicosapentaenoic acid (EPA), and combinations thereof.

The concentration and relative amounts of protein, carbohydrate, and/or fat in the exemplary nutritional compositions can vary considerably depending upon, for example, the specific dietary needs of the intended user. In a specific embodiment, the nutritional composition comprises protein in an amount of about 2 wt % to 15 wt %, carbohydrate in an amount of about 5 wt % to 25 wt %, and fat in an amount of about 0.5 wt % to 12 wt %, based on the weight of the nutritional composition.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

The nutritional composition may further comprise one or more additional components that may modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

Additionally, the nutritional composition may further include vitamins or related nutrients, non-limiting examples of which include vitamin A, vitamin B12, vitamin C, vitamin D, vitamin K, thiamine, riboflavin, pyridoxine, niacin, folic acid, pantothenic acid, biotin, choline, inositol, salts and derivatives thereof, and combinations thereof. Water soluble vitamins may be added in the form of a water-soluble vitamin (WSV) premix and/or oil-soluble vitamins may be added in one or more oil carriers as desired.

In additional embodiments, the nutritional composition may further include one or more minerals, non-limiting examples of which include calcium, phosphorus, magnesium, zinc, manganese, sodium, potassium, molybdenum, chromium, chloride, and combinations thereof.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The intact exosomes may be added at any time as desired in the process, for example, to the aqueous solution or to the emulsified blend. The intact exosomes may be dry blended in powder form with one or more dry ingredients, for example, for combined addition to a liquid composition or if a powdered nutritional product is desirable.

The methods of reducing muscle atrophy and/or increasing muscle regeneration comprise administering a nutritional composition as described herein to the subject at risk of experiencing of muscle atrophy. The nutritional composition may be administered in powder or liquid form, as desired. In specific embodiments, the nutritional compositions comprising bovine milk-isolated exosomes are administered to a subject once or multiple times daily or weekly. In specific embodiments, the nutritional composition is administered to the subject from about 1 to about 6 times per day or per week, or from about 1 to about 5 times per day or per week, or from about 1 to about 4 times per day or per week, or from about 1 to about 3 times per day or per week. In specific embodiments, the nutritional composition is administered once or twice daily for a period of at least one week, at least two weeks, at least three weeks, or at least four weeks.

In specific embodiments, the nutritional compositions are administered in an effective amount to reduce muscle atrophy and/or increase muscle regeneration. In additional specific embodiments, the nutritional compositions are administered in an amount sufficient to provide a dosage of from about 0.01 to about 10 g of bovine milk-isolated exosomes. In additional embodiments, a dosage of from about 0.1 to about 10 g, or from about 1 to about 5 g, of bovine milk-isolated exosomes are administered to a subject via administration of the nutritional composition.

The following Examples demonstrate aspects of the inventive methods and are provided solely for the purpose of illustration. The Examples are not to be construed as limiting of the general inventive concepts, as many variations thereof are possible without departing from the spirit and scope of the general inventive concepts.

### Example 1

This examples describes preparation of powdered exosomes. Exosomes first were isolated from bovine milk and then dried. Specifically, raw unprocessed bovine milk was aliquoted and immediately frozen at −80° C. Aliquots were thawed on ice and subjected to a first centrifugation at about 12,000 G at about 4° C. for about 15 minutes to obtain a whey layer formed between a top layer of fat (lipid) and the cell debris pellet. The whey fraction was transferred to a clean tube and centrifuged two more times, each at about 21,000 G and about 4° C. for about 30 minutes, conditions which maintain the exosomes in intact form, to remove additional fat and debris. A substantially clear whey fraction was obtained. The substantially clear whey fraction was microfiltered using a 0.22 μm filter of hydrophilic polyethersulfone, and the microfiltered whey was then centrifuged at 100,000 G at 4° C. for about 60 minutes to obtain an exosome-containing pellet. The exosome-containing pellet was carefully suspended in sterile PBS (137 mM NaCl, 2.7 mM KCl, 8 mM Na₂HPO₄, and 2 mM KH₂PO₄; pH 7.4) or sterile molecular biology grade water in a centrifugation tube, which was then incubated for 12-36 hours in an orbital shaker at 4° C. and 150 rpm. Notably, when the orbital shaking step was not utilized, aggregates appeared that required extensive pipetting to break up, leading to exosome membrane damage.

The exosomes were first frozen at −80° C. for at least 2 hours and the frozen exosomes were then freeze dried under conditions which maintain the exosomes in intact form. Specifically, the step of freeze-drying comprised exposing the frozen exosomes to a temperature of −80° C. and a vacuum of less than 0.3 mbar for a sufficient time period, about 24 hours, to ensure a low moisture content. The resulting product comprised powdered exosomes.

Importantly, thawing the exosomes prior to the vacuum stage of the freeze-drying led to milk exosome membrane damage. Therefore, embodiments of methods described herein comprise freezing the milk exosomes and keeping the milk exosomes frozen until freeze-drying is complete and powdered milk exosomes result. The isolation and drying conditions described herein maintain the integrity of the exosome lipid membrane and the bioactivity of the contents of the exosomes.

### Example 2

A first portion of the powdered intact bovine milk-isolated exosomes prepared in Example 1 were dissolved in water. A second portion of the powdered exosomes prepared in Example 1 were dissolved in water and placed in an Ultrasons P-selecta sonifier for 1 hour. To ensure complete disruption of the milk exosome membrane, the sonicated milk exosomes were incubated for 15 minutes at 95° C. after sonication.

The exosomes were used in vitro assays to assess their ability to influence mechanisms leading to muscle atrophy and muscle regeneration. The in vitro experiments were performed with L6.C11 rat skeletal muscle myoblast cell line (ECACC No. 92102119). This cell line was grown in DMEM (Dulbecco's Modified Eagle Medium) culture medium supplemented with 10% (v/v) fetal bovine serum (FBS), 2 mmol/L glutamine, 100 units/ml penicillin, and 0.1 mg/ml streptomycin in an atmosphere of 5% CO₂ and 95% air, and was maintained at sub-confluent densities in the growth media. Cells were differentiated into myotubes by culturing them for 5 days in DMEM containing 2% FBS (v/v).

### Dexamethasone-Induced Protein Degradation

To evaluate the ability of the bovine milk-isolated exosomes to reduce muscle atrophy/wasting, protein degradation was first assessed in the presence or absence of the intact milk exosomes. Dexamethasone, a glucocorticoid which triggers protein degradation in muscle, was used to mimic hypercatabolic conditions. The L6.C11 rat skeletal muscle myoblasts were differentiated into myotubes (5 days in differentiation media) and were labeled with 1 μCi/ml of L-[ring-3,5-3H]-tyrosine for 48 h in DMEM culture medium plus 10% FBS, with 15 μg/ml of the intact bovine milk-isolated exosomes or the sonicated exosomes, respectively. Cells were rinsed once with PBS-Tyr and then placed in DMEM supplemented with 10% FBS, 2 mM L-tyrosine plus 5 μM DEX (dexamethasone, degradation medium) for 2 h to allow degradation of very short-lived proteins. The cells were then rinsed twice with PBS-Tyr and fresh degradation medium was added. Cells were incubated for an additional 24 h period in the degradation medium. At the end of the incubation period, to measure degradation rates, the culture medium was transferred to a microcentrifuge tube containing 100 μl of bovine serum albumin (BSA) (10 mg/ml). Trichloroacetic acid (TCA) was added to a final concentration of 10% (w/v). After incubation at 4° C. for at least 1 h, samples were centrifuged for 5 min. The precipitates were then dissolved with tissue solubilizer. Cell monolayers were washed with ice-cold (PBS) and solubilized with 0.5 M NaOH containing 0.1% Triton X-100. Radioactivity was measured using a Beckman LS6000 SE scintillation counter. Protein degradation was expressed as the percentage protein degraded over a 24-h period.

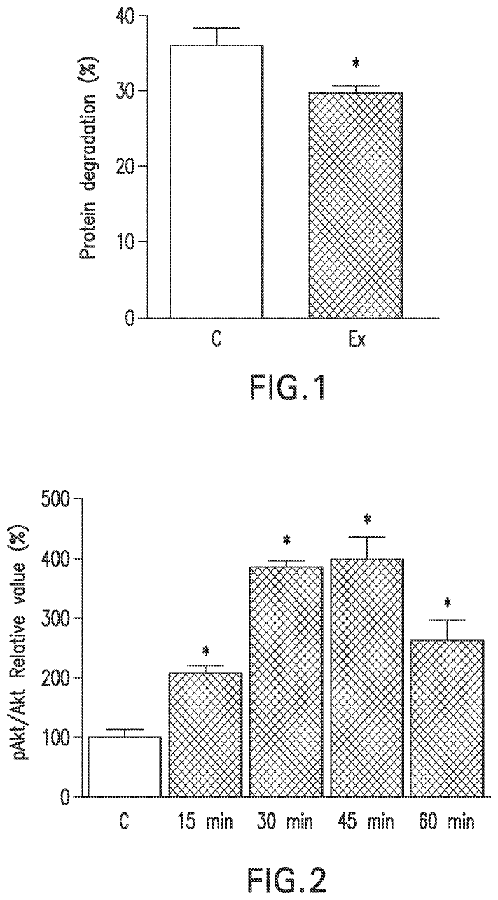

As shown in FIG. 1, compared to a control (C), in which no exosomes were added, when muscle cells were incubated in the presence of dexamethasone, addition of the intact bovine milk-isolated exosomes (Ex) attenuated, in a statistically significant manner, protein degradation induced by dexamethasone. This is a particularly relevant result given that muscle growth reflects the balance between anabolic and catabolic processes. Therefore, the intact bovine milk-isolated exosomes are useful to treat or prevent conditions that are characterized by having increased protein degradation rates.

### Molecular Markers of Protein Degradation and Expression

Another set of experiments was conducted to demonstrate the effects of the intact bovine milk-isolated exosomes and, in some cases, sonicated milk exosomes on key molecular markers, specifically, for muscle atrophy, the ubiquitin-proteasome pathway markers Akt, FoxO, ubiquitin and atrogin-1, and for muscle regeneration and myogenesis, the marker Mef2.

To study the phosphorylation status of the proteins involved in signaling events, L6 myotubes prepared as described previously were incubated with 15 μg/ml of the intact bovine milk-isolated exosomes for 15, 30, 45 and 60 minutes. Plates were flash frozen in liquid nitrogen and processed for protein extract preparation. Cells were scraped with 400 μl/60-mm plate of cold 30 mM Tris-HCl, pH 7.4, 25 mM NaCl, 1% (v/v) Triton X-100, 0.1% sodium dodecyl sulfate (SDS), 10 mM sodium fluoride, 10 mM sodium pyrophosphate, 1 mM sodium orthovanadate, 1 mM ethyleneglycol-bis(β-aminoethyl)-N,N,N′,N′-tetraacetic acid (EGTA), 20 nM okadaic acid, 10 μg/ml aprotinin, 10 μg/ml leupeptin, 10 μg/ml pepstatin. After 10 min on ice, extracts were centrifuged for 10 min at 4° C. at 13,000 G. The protein concentration was measured using the bicinchoninic acid method. Proteins were separated by SDS-PAGE, transferred to nitrocellulose membranes (Schleicher & Schüll), and immunoblotted with selected antibodies. The immunoblots were developed using an enhanced chemiluminescence detection system (Amersham Biosciences), following the manufacturer's instructions.

To study the expression of proteins associated with protein degradation, FoxO, ubiquitin and atrogin-1, L6 myotubes, prepared as described, were pre-incubated for 48 h with 15 μg/ml of the intact bovine milk-isolated exosomes or sonicated milk exosomes, respectively, and then incubated for 24 h with 5 μM dexamethasone (DEX) in the presence or absence of effectors. When 100 nM Bafilomycin A1 was used, it was added concomitantly with DEX and remained in the culture throughout the treatment. After treatment, cells were lysed with RIPA buffer supplemented with phosphatase and protease inhibitors, 10 mM sodium fluoride, 10 mM sodium pyrophosphate, 1 mM sodium ortho vanadate, 1 mM EGTA, 20 nM okadaic acid, 10 μg/ml aprotinin, 10 μg/ml leupeptin and 10 μg/ml pepstatin. The protein concentration was measured using the bicinchoninic acid method. Proteins (40 μg) were separated by SDS-PAGE, transferred onto nitrocellulose membranes, and immunoblotted with specific antibodies; the immunoblots were developed by an enhanced chemiluminescence detection method.

To study the expression of MEF2 during the differentiation process, L6 myoblasts were treated with 15 μg/ml of the intact bovine milk-isolated exosomes or the sonicated exosomes for 3 days. Cells were processed as above, with RIPA and inhibitors of phosphatases.

The protein kinase Akt is one of the main regulators of protein degradation and subsequent muscle atrophy. That process, which involves the shrinkage of myofibers due to net loss of proteins, organelles and cytoplasm, mainly occurs through the ubiquitin-proteasome pathway. Under anabolic conditions, the ubiquitin-proteasome pathway is blocked by phosphorylated Akt through the inactivating phosphorylation of Forkhead box 0 (FoxO) transcription factor. The effect of incubation with the intact bovine milk-isolated exosomes on Akt phosphorylation status is shown in FIG. 2. When myotubes where incubated with the intact bovine milk-isolated exosomes, Akt phosphorylation significantly increased as compared to a control (C) free of the exosomes.

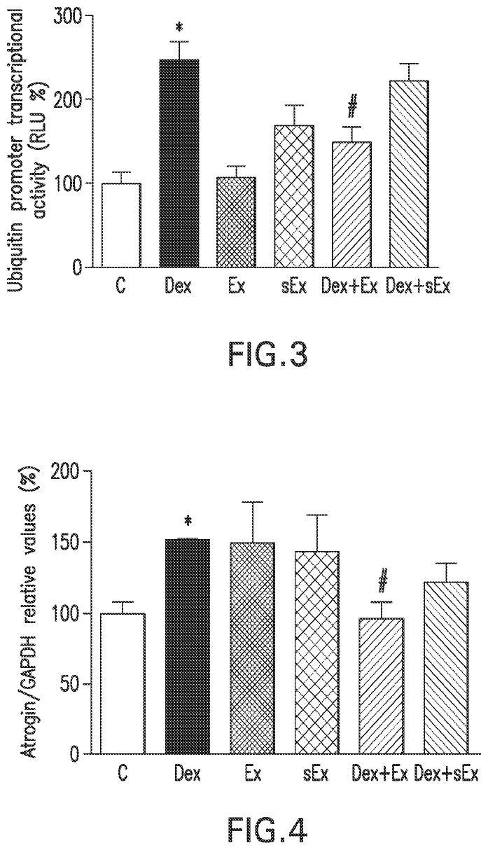

Within the ubiquitin-proteasome degradative pathway, two elements are considered to be key effectors of muscle atrophy. The first one is the ubiquitin gene which, under degradative conditions, is induced to produce the small regulatory ubiquitin protein. Proteins to be degraded are tagged with chains of ubiquitin, a monomeric 76 amino acid protein that becomes the signal that directs the ubiquitinylated protein to the proteasome. That reaction is catalyzed by two ubiquitin-ligases, atrogin and Murfl. Increased levels of both ubiquitin and ubiquitin ligases are required to trigger ubiquitin-proteasome-mediated protein degradation. FIG. 3 shows that incubation with dexamethasone (Dex) induced ubiquitin promoter transcriptional activity. Dexamethasone-induced transcriptional activity of ubiquitin promoter is a hallmark of protein degradation. Such an induction of transcriptional activity was not seen when myotubes were incubated with either the intact bovine milk-isolated exosomes (Ex) alone or the sonicated milk exosomes (sEx) alone, thereby indicating that neither the intact bovine milk-isolated exosomes nor the sonicated milk exosomes activate per se the ubiquitin-proteasome degradative pathway. When cells were treated with dexamethasone and the intact bovine milk-isolated exosomes (Dex+Ex), ubiquitin promoter transcriptional activity was measured at levels found in the control where no dexamethasone was added. Thus, the freeze dried exosomes protected against the dexamethasone-induced ubiquitin promoter transcriptional activity. Remarkably, that protective effect against protein degradation was not seen when cells were treated with dexamethasone and the sonicated exosomes (Dex+sEx) in which the membrane was disrupted.

In addition to increasing ubiquitin gene transcription, the activation of the ubiquitin-proteasome pathway requires compartment-specific ubiquitin ligase complexes that mark substrates with ubiquitin for proteasome degradation. Accordingly, protein levels of atrogin-1, a muscle specific ubiquitin ligase, in myotubes were determined. As shown in FIG. 4, dexamethasone (Dex) markedly increased atrogin-1 expression levels, but neither the intact bovine milk-isolated exosomes (Ex) alone nor the sonicated milk exosomes (sEx) alone statistically significantly increased atrogin-1 expression levels as the respective p-value was higher than 0.05 and therefore, statistically, no conclusion that bovine milk-isolated exosomes (Ex) or the sonicated milk exosomes (sEx) are different from the control (C). Only Dex was statistically different from the control (C). As further shown in FIG. 4, incubation of myotubes with dexamethasone and the intact bovine milk-isolated exosomes (Dex+Ex) prevented the dexamethasone-induced increase in atrogin-1 levels. However, incubation of myotubes with dexamethasone and the sonicated exosomes (Dex+sEx) did not provide this protective effect. Thus, the increase in the atrogin-1 protein level, which is required for protein degradation through the ubiquitin-proteasome system, was prevented with the intact bovine milk-isolated exosomes.

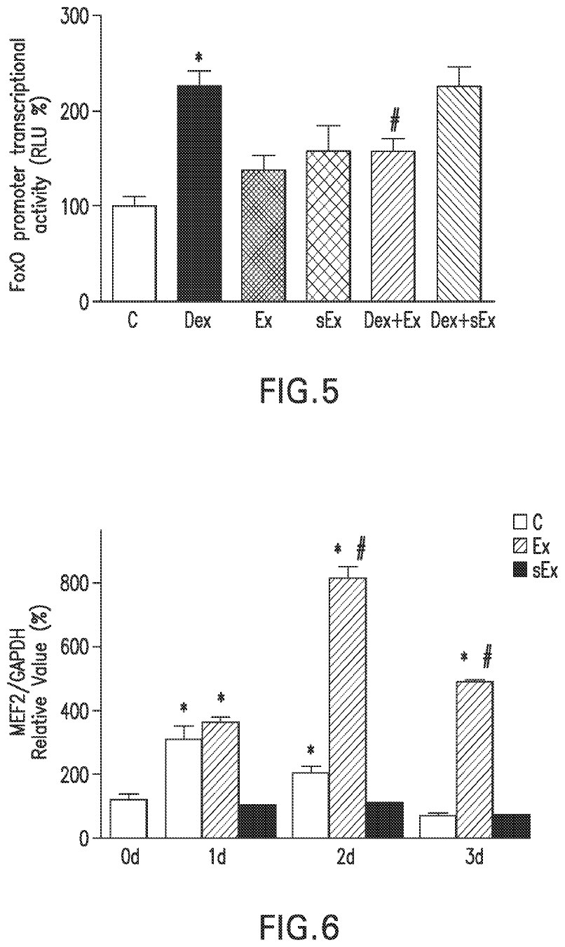

As described previously, the ubiquitin-proteasome pathway is blocked by phosphorylated Akt through the inactivating phosphorylation of FoxO transcription factor. Accordingly, FoxO gene transcription was also assessed. FoxO is a master regulator that controls the ubiquitin-proteasome catabolic pathway and its phosphorylation is required to increase protein degradation. As shown in FIG. 5, when myotubes were incubated with dexamethasone (Dex) alone, FoxO promoter transcriptional activity was significantly induced. Incubation with either the intact bovine milk-isolated exosomes (Ex) alone or the sonicated exosomes (sEx) alone did not increase the FoxO promoter transcriptional activity. This is again of particular relevance, given that it shows neither the intact bovine milk-isolated exosomes alone nor the sonicated freeze-dried exosomes alone trigger protein degradation per se. When myotubes were incubated with dexamethasone and the intact bovine milk-isolated exosomes (Dex+Ex), transcriptional activity of the FoxO promoter was significantly decreased. Remarkably, that protective effect was not shown when myotubes were incubated with dexamethasone and sonicated milk exosomes (Dex+sEx). Thus, dexamethasone-induced transcriptional activity of the protein degradation-promoter factor FoxO was abolished by the intact bovine milk-isolated exosomes, but not by the sonicated milk exosomes.

Effects of the intact bovine milk-isolated exosomes on muscle hypertrophy or differentiation were also studied, specifically by measuring Mef2 levels. Mef2 increases during the differentiation of muscle cells to yield mature myotubes, which is required for muscle growth and regeneration. As shown in FIG. 6, when L6 myoblasts were transferred to differentiation medium (C), Mef2 expression transiently increased from the level at day 0, particularly at days 1 and 2. As also shown in FIG. 6, the addition of the intact bovine milk-isolated exosomes to the differentiation medium (Ex) significantly increased the expression of Mef2 over all three days. That induction was not achieved when sonicated exosomes were added to the differentiation medium (sEx).

Taken together, these results indicate that (1) the intact bovine milk-isolated exosomes do not induce protein degradation or muscle wasting by themselves; (2) in muscle wasting conditions, the intact bovine milk-isolated exosomes are able to decrease protein degradation by inhibiting the ubiquitin-proteasome pathway; (3) the intact bovine milk-isolated exosomes are able to significantly increase the Mef2 marker of muscle regeneration and differentiation; and (4) these effects provided by the intact bovine milk-isolated exosomes are not provided by sonicated bovine milk exosomes in which the membrane is disrupted. Thus, the intact bovine milk-isolated exosomes both protect muscle cells from protein degradation, thereby reducing muscle atrophy, and significantly increase the Mef2 marker of muscle regeneration and differentiation, thereby promoting muscle regeneration.

The intact bovine milk-isolated exosomes therefore provide a novel tool to promote muscle anabolism over muscle catabolism, reducing muscle atrophy and promoting muscle regeneration in subjects having or at risk of developing diseases or conditions that involve muscle wasting.

While the present application has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and processes, or illustrative examples shown and described. Accordingly, departures may be made from such details without departing from the spirit or scope of the general inventive concept.

1. A method of decreasing muscle atrophy and/or promoting muscle regeneration in a subject at risk of muscle atrophy, the method comprising orally administering a nutritional composition comprising at least one of protein, fat and carbohydrate, and bovine milk-isolated exosomes comprising intact exosomes.

2. The method of claim 1, wherein greater than 90% of the bovine milk-isolated exosomes are from about 10 nanometers to about 250 nanometers in diameter.

3. The method of claim 1, wherein the nutritional composition comprises from about 0.001 to about 10 wt % of the bovine milk-isolated exosomes, based on the weight of the nutritional composition.

4. The method of claim 1, wherein the subject is a human adult over 40 years of age.

5. The method of claim 1, wherein the subject suffers from malnutrition, acquired immune deficiency syndrome (AIDS), cancer, diabetes, chronic obstructive pulmonary disease (COPD), amyotrophic lateral sclerosis (ALS), non-alcoholic fatty liver disease (NAFLD), or a burn injury, or has undergone clinical corticosteroid treatment.

6. The method of claim 1, for decreasing muscle atrophy.

7. The method of claim 1, for promoting muscle regeneration.

8. The method of claim 1, wherein the nutritional composition comprises protein.

9. The method of claim 8, wherein the nutritional composition further comprises fat and carbohydrate.

10. The method of claim 1, wherein the nutritional composition comprises at least one protein selected from whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hamp proteins, flax seed proteins, earthworm proteins, insect proteins, and combinations of two of more thereof.

11. The method of claim 1, wherein the nutritional composition comprises at least one fat selected from algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, long chain polyunsaturated fatty acids, and combinations of two of more thereof.

12. The method of claim 1, wherein the nutritional composition comprises at least one carbohydrate selected from maltodextrin, hydrolyzed starch, modified starch, hydrolyzed cornstarch, modified cornstarch, polydextrose, dextrins, corn syrup, corn syrup solids, rice maltodextrin, brown rice mild powder, brown rice syrup, sucrose, glucose, fructose, lactose, high fructose corn syrup, honey, maltitol, erythritol, sorbitol, isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, fiber, fruit puree, vegetable puree, isomalto-oligosaccharides, monosaccharides, disaccharides, tapioca-derived carbohydrates, inulin, and artificial sweeteners, and combinations of two of more thereof.

13. The method of claim 1, wherein the nutritional composition comprises from about 1 wt % to about 30 wt %, from about 1 wt % to about 25 wt %, from about 1 to about 20 wt %, from about 1 to about 15 wt %, from about 1 to about 10 wt %, or from about 10 wt % to about 30 wt % protein, based on the weight of the nutritional composition.

14. The method of claim 1, wherein the nutritional composition comprises from 0.5 wt % to 20 wt %, from about 0.5 to about 15 wt %, from about 0.5 to about 10 wt %, from about 0.5 to about 5 wt %, or from about 5 to about 15 wt % fat, based on the weight of the nutritional composition.

15. The method of claim 1, wherein the nutritional composition comprises from about 5 wt % to about 75 wt %, from about 5 wt % to about 70 wt %, from about 5 wt % to about 65 wt %, from about 5 wt % to about 50 wt %, from about 5 wt % to about 40 wt %, from about 5 wt % to about 30 wt %, from about 5 wt % to about 25 wt %, from about 10 wt % to about 65 wt %, from about 20 wt % to about 65 wt %, from about 30 wt % to about 65 wt %, from about 40 wt % to about 65 wt %, or from about 15 wt % to about 25 wt % carbohydrate, based on the weight of the nutritional composition.

16. The method of claim 1, wherein the nutritional composition is a liquid nutritional composition and comprises from about 1 to about 15 wt % of protein, from about 0.5 to about 10 wt % fat, and from about 5 to about 30 wt % carbohydrate, based on the weight of the nutritional composition.

17. The method of claim 1, wherein the nutritional composition is a powder nutritional composition and comprises from about 10 to about 30 wt % of protein, from about 5 to about 15 wt % fat, and from about 30 wt % to about 65 wt % carbohydrate, based on the weight of the nutritional composition.

18. The method of claim 1, wherein the nutritional composition comprises at least one protein comprising milk protein concentrate and/or soy protein isolate, at least one fat comprising canola oil, corn oil, coconut oil and/or marine oil, and at least one carbohydrate comprising maltodextrin, sucrose, and/or short-chain fructooligosaccharide.
